(19) United States
(12) Patent Application Publication
(10) Pub. No.: US 2026/0335201 A1
(43) Pub. Date: Oct. 29, 2026

(54) CLEANING AGENT COMPOSITION FOR ANIMAL AND METHOD FOR CLEANING ANIMAL

(71) Applicant: Animal Hospital KYOTO, Inc., Kyoto-shi, Kyoto (JP)

(72) Inventors: Yuzo SONODA, Kyoto-shi, Kyoto (JP); Hisaki TANAKA, Kyoto-shi, Kyoto (JP)

(21) Appl. No.: 19/121,292

(22) PCT Filed: Nov. 10, 2023
(86) PCT No.: PCT/JP2023/040515

§ 371 Date: Apr. 15, 2025

(30) Foreign Application Priority Data
Nov. 28, 2022 (JP) 2022-188842

(51) Int. Cl. A61K 8/25; A61Q 5/02

(52) U.S. Cl. CPC A61K 8/25; A61Q 5/02; A61K 2800/33

(57) ABSTRACT

[Summary]

[Problem to Be Solved] To provide a composition and a cleansing method that do not require running water and allow for the simple and effective removal of oil-based contamination.

[Solution] The composition of the present invention comprises calcium sodium phosphosilicate in an amount of 0.01 mass parts or more and 10.0 mass parts or less, and water. The calcium sodium phosphosilicate includes a bioactive glass containing silicon dioxide, calcium oxide, sodium oxide, and phosphorus pentoxide, and a bioactive glass extract obtained by removing the bioactive glass from a bioactive glass-containing liquid comprising the bioactive glass and an aqueous solvent. By applying the above composition to a cleansing target site of an animal and wiping off the applied composition, oil-based contamination can be effectively removed without requiring a water rinsing step.

### TECHNICAL FIELD

The present invention relates to a cleansing agent composition for animals and a method for cleansing animals.

### BACKGROUND ART

Cleansing of the body of animals (primarily dogs and cats) is generally performed in the owner's home and/or at a grooming salon. Since a large portion of an animal's body is covered with fur, not only does the cleansing operation with shampoo require time and effort, but the drying operation after shampooing also takes time and effort, imposing a significant burden on the owner or grooming salon who take care of the animal.

Furthermore, prolonged cleansing and drying operations for the animal impose a significant burden on the animal's body, and in the case of elderly dogs or other animals with underlying conditions such as heart disease, cleansing operations using shampoo are considered contraindicated. As a result, this leads to issues such as animal odor, skin diseases, and dandruff.

In the case of dogs and cats, unlike humans, apocrine glands and sebaceous glands are present throughout the entire body, so epidermal lipids are not cleansed by sweat (sweat containing water, as in humans, from eccrine glands), and oil-based contamination caused by sebum accumulated on the skin and fur is the primary type of dirt.

As a method for cleansing such oil-based contamination, in addition to shampoos containing synthetic surfactants similar to those used by humans, oil cleansing products and the like have been marketed (see Patent Literature 1).

### CITATION LIST

### Patent Literature

[Patent Literature 1] JP2010-077097A

### SUMMARY OF THE INVENTION

### Problems to be Solved by the Invention

However, even with oil cleansing, washing with running water and drying are still required; thus, the burden on the owner or grooming salon in terms of labor, as well as the physical burden on the animal, is not alleviated. Accordingly, there is a demand for an animal cleansing agent that does not require running water or similar means and that enables the simple and effective removal of oil-based contamination, along with a composition and a cleansing method that enable such removal.

The objective of the present invention is to provide a composition and a cleansing method that do not require running water and allow for the simple and effective removal of oil-based contamination.

### Means for Solving the Problems

As a result of extensive studies aiming to solve the above problem, the inventors of the present invention have found that by using calcium sodium phosphosilicate, the above objective can be achieved, leading to the completion of the present invention. Specifically, the present invention provides the following:

The invention according to the first aspect provides a cleansing agent composition for animals comprising calcium sodium phosphosilicate in an amount of 0.01 mass parts or more and 10.0 mass parts or less, and water.

According to the invention of the first aspect, the emulsifying action of the calcium sodium phosphosilicate suspension enables the removal of oil-based contamination and odor from animals. Additionally, it effectively removes discoloration stains (oil-based contamination and protein-based contamination) around the ears, eyes, and mouth. Furthermore, since it suppresses the proliferation of bacteria associated with oil-based contamination, it can prevent conditions such as pyoderma caused by Staphylococcus aureus and dermatitis caused by Malassezia (fungus).

For cleansing animals, the composition may be applied to the cleansing target site of the animal and then wiped off. Accordingly, as it does not require rinsing with running water, the physical burden on the animal is reduced, making it suitable for use on elderly dogs or other animals with underlying conditions such as heart disease. Moreover, it reduces the labor burden on owners and grooming salons, allowing for daily use of the composition.

Thus, according to the invention of the first aspect, it is possible to provide a composition that does not require running water and that allows for the simple and effective removal of oil-based contamination.

The invention according to the second aspect is the invention according to the first aspect, wherein the calcium sodium phosphosilicate comprises a bioactive glass containing silicon dioxide, calcium oxide, sodium oxide, and phosphorus pentoxide, and a bioactive glass extract obtained by removing the bioactive glass from a bioactive glass-containing liquid comprising the bioactive glass and an aqueous solvent.

According to the invention of the second aspect, the extract contains sodium ions and calcium ions. These ions act on the acid-base bonds of skin proteins and weaken the intermolecular (intramolecular) bonds of proteins. As a result, the skin softens, improving the permeability of moisturizers and other substances.

Furthermore, since sodium ions and calcium ions are moisturizing components present in keratinocytes (NMF: natural moisturizing factors), the sodium ions and calcium ions supplied from calcium sodium phosphosilicate also function like the skin's inherent moisturizing components.

Additionally, as the extract exhibits alkalinity, it exerts a bactericidal effect. Thus, it more effectively suppresses the proliferation of bacteria associated with oil-based contamination, preventing conditions such as pyoderma caused by Staphylococcus aureus and dermatitis caused by Malassezia (fungus).

The invention according to the third aspect is the invention according to the first or second aspect, wherein the composition has a pH of 8 or more and less than 13.

According to the invention of the third aspect, since the extract exhibits alkalinity, it exerts a bactericidal effect. Thus, it more effectively suppresses the proliferation of bacteria associated with oil-based contamination.

The invention according to the fourth aspect is the invention according to any one of the first to third aspects, wherein the composition substantially does not contain synthetic surfactants.

According to the invention of the fourth aspect, irritation to the cleansing target site of the animal can be reduced. Additionally, it alleviates the burden on the skin of grooming salon staff, who are frequently exposed to cleansing agents.

The invention according to the fifth aspect is the invention according to any one of the first to fourth aspects, wherein the composition further comprises a naturally derived amphoteric surfactant.

Amphoteric surfactants cause less irritation and are safer than anionic and cationic surfactants. Although amphoteric surfactants alone do not provide sufficient cleansing efficacy, according to the invention of the fifth aspect, the combination of amphoteric surfactants and calcium sodium phosphosilicate enables high cleansing power while maintaining safety.

The invention according to the sixth aspect is the invention according to any one of the first to fifth aspects, comprising a method for cleansing animals, including a step of applying the composition to a cleansing target site of the animal and a step of wiping off the applied composition, wherein the method does not require a water rinsing step.

### Effect of the Invention

According to the present invention, it is possible to provide a composition and a cleansing method that do not require running water and that allow for the simple and effective removal of oil-based contamination.

### MODES FOR CARRYING OUT THE INVENTION

Hereinafter, a description of an example of a preferred mode for carrying out the present invention is provided. It should be noted that this is merely an example and that the technical scope of the present invention shall not be limited thereto.

### <Animal Cleansing Agent Composition>

The animal cleansing agent composition according to the present embodiment comprises calcium sodium phosphosilicate in an amount of 0.01 mass parts or more and 10.0 mass parts or less, and water.

### {Calcium Sodium Phosphosilicate}

Calcium sodium phosphosilicate may be a bioactive glass-containing liquid comprising 0.01% by weight to 10.0% by weight of bioactive glass and 99.99% by weight to 90.0% by weight of an aqueous solvent; or it may be a bioactive glass extract obtained by removing the bioactive glass from said liquid.

### [Bioactive Glass]

Bioactive glass is a powdered glass material, comprising silicon dioxide, sodium oxide, calcium oxide, and phosphorus pentoxide. Bioactive glass is activated by moisture, releasing ions that are components with high bioavailability (extractable components from bioactive glass). In particular, the sustained release of sodium ions acts on the acid-base bonds of skin proteins, weakening intermolecular (intramolecular) protein bonds. As a result, the skin softens, improving the permeability of moisturizers and other substances.

Additionally, since sodium ions and calcium ions are moisturizing components within keratinocytes (NMF: natural moisturizing factors), the sodium ions and calcium ions supplied from calcium sodium phosphosilicate also function as the skin's inherent moisturizing components.

Furthermore, due to the emulsifying action of the calcium sodium phosphosilicate suspension, it can remove oil-based contamination and odor from animals. Additionally, it effectively removes discoloration stains (oil-based contamination and protein-based contamination) around the ears, eyes, and mouth. Furthermore, since it suppresses the proliferation of bacteria associated with oil-based contamination, it can prevent conditions such as pyoderma caused by Staphylococcus aureus and dermatitis caused by Malassezia (fungus).

Additionally, as the extract exhibits alkalinity, it exerts a bactericidal effect. Thus, it more effectively suppresses the proliferation of bacteria associated with oil-based contamination, preventing conditions such as pyoderma caused by Staphylococcus aureus and dermatitis caused by Malassezia (fungus).

The bioactive glass is not particularly limited as long as it is a glass material that releases alkaline ions and has degradability. Examples of bioactive glass include fluoroaluminosilicate glass used in dental materials and Vitryxx by SCHOTT.

The primary average particle size of the bioactive glass is not particularly limited. From the viewpoint of facilitating the extraction of active ingredients, the primary average particle size of the bioactive glass is preferably 0.5 μm or more, and more preferably 1.0 μm or more.

On the other hand, from the viewpoint of extracting a greater amount of active ingredients, the primary average particle size of the bioactive glass is preferably 15.0 μm or less, more preferably 10.0 μm or less, even more preferably 8.0 μm or less, and most preferably 5.0 μm or less.

### [Water-Based Solvent]

A water-based solvent is not particularly limited as long as it can extract the active ingredients of the animal cleansing agent composition containing bioactive glass without compromising their function as active ingredients and while minimizing damage to the skin; it may be water, a water-miscible solvent, or a mixed solvent thereof.

Water includes any types that remains in a liquid state at room temperature, such as purified water, ion-exchanged water, and tap water.

Examples of water-miscible solvents include C1-4 alcohols such as methanol, ethanol, n-propanol, isopropanol, n-butanol, s-butanol, isobutanol, and t-butanol; ketone-based solvents such as acetone; amide-based solvents such as dimethylformamide and dimethylacetamide; sulfoxide-based solvents such as dimethyl sulfoxide; nitrile-based solvents such as acetonitrile; and organic acid solvents such as acetic acid. However, in the present embodiment, since it is necessary to reduce damage to the skin, the water-miscible solvent may be ethanol or isopropanol, preferably ethanol.

### [Water-Soluble Components]

The water-based solvent may contain water-soluble components as long as they do not compromise the effects of the invention described in the present embodiment. In the present embodiment, the overall composition preferably has a pH of 8 or more and less than 13. If the composition has a pH of 8 or more, it can effectively suppress the proliferation of bacteria associated with oil-based contamination. Additionally, if the composition has a pH of less than 13, it can reduce the hydrolysis of active ingredients due to alkalinity and mitigate the potential damage that alkalinity may cause to the skin of the subject.

Since calcium sodium phosphosilicate itself has a pH-buffering function, it can maintain a pH of 8 or more and less than 13 without the need for strict control of its blending amount, unlike alkaline components such as sodium hydroxide. However, to enhance pH stability, the composition may contain a pH-buffering component as a water-soluble component.

Examples of weakly alkaline buffer solutions include Tris buffer (tris(hydroxymethyl)aminomethane), TE buffer (Tris buffer with added EDTA (ethylenediaminetetraacetic acid)), and ammonium chloride buffer solution.

Other water-soluble components include additives commonly used in cosmetics and quasi-drugs, such as powders, humectants, thickeners, and preservatives.

### [Ratio of Bioactive Glass to Water-Based Solvent in Bioactive Glass-Containing Liquid]

The ratio of bioactive glass to water-based solvent in the bioactive glass-containing liquid is not particularly limited as long as the bioactive glass extract effectively exhibits its function as an animal skin external preparation.

As a guideline, to effectively extract the active components contained in the bioactive glass, the bioactive glass content is preferably 0.01% by weight or more relative to 100% by weight of the bioactive glass-containing liquid. In other words, the water-based solvent content is preferably 99.99% by weight or less relative to 100% by weight of the bioactive glass-containing liquid.

Additionally, to obtain a sufficient amount of bioactive glass extract (filtrate) when the bioactive glass is removed from the bioactive glass-containing liquid, the bioactive glass content is preferably 10.0% by weight or less relative to 100% by weight of the bioactive glass-containing liquid. In other words, the water-based solvent content is preferably 90.0% by weight or more relative to 100% by weight of the bioactive glass-containing liquid.

### [Content of Calcium Sodium Phosphosilicate]

In the present embodiment, the content of calcium sodium phosphosilicate is not particularly limited. From the viewpoint of maintaining a pH of 8 or higher, promoting the emulsifying function of oil-based contamination as a sebum remover, and effectively suppressing the proliferation of bacteria associated with oil-based contamination, the content of calcium sodium phosphosilicate in the composition is preferably 0.01 mass parts or more, more preferably 0.5 mass parts or more, even more preferably 1.0 mass parts or more, still more preferably 1.5 mass parts or more, and particularly preferably 2.0 mass parts or more.

Additionally, from the viewpoint of keeping the pH below 13, the content of calcium sodium phosphosilicate in the composition is preferably 10.0 mass parts or less, from the viewpoint of keeping the pH below 12.5, it is more preferably 8.0 mass parts or less, and from the viewpoint of keeping the pH below 12, it is even more preferably 4.0 mass parts or less. Since the composition has a pH of less than 13, it is possible to reduce the hydrolysis of active ingredients due to alkalinity and mitigate potential damage that alkalinity may cause to the subject's skin.

### [Method for Producing Calcium Sodium Phosphosilicate]

Calcium sodium phosphosilicate is obtained by mixing 0.01% by weight to 10.0% by weight bioactive glass with 99.99% by weight to 90.0% by weight water-based solvent, allowing the mixture to stand for 1 minute to 100,000 hours and then removing the bioactive glass from the bioactive glass-containing liquid.

The method for mixing bioactive glass and a water-based solvent is not particularly limited, and examples include mixing using equipment such as an ultrasonic oscillator, shaker, mixer, or stirrer.

The method for removing bioactive glass from the bioactive glass-containing liquid is not particularly limited, and examples of removal methods include filtration. The method of filtration is not particularly limited. One embodiment includes subjecting the bioactive glass-containing liquid to suction filtration using a filter with a pore size smaller than the average particle size of the bioactive glass.

Although not essential, the pH of the filtrate may be checked, and if necessary, the pH of the filtrate may be adjusted to 8 or more and less than 12.

### {Water}

The type of water is not particularly limited and includes any form that remains in a liquid state at room temperature, such as purified water, ion-exchanged water, and tap water.

### {Surfactants}

Although not essential, it is preferable that the composition described in the present embodiment substantially does not contain synthetic surfactants. In the present embodiment, “substantially does not contain” refers to an amount that does not cause skin damage when the cleansing agent composition is used as a shampoo or similar product. This allows for the reduction of irritation to the cleansing target site of the animal. Additionally, it alleviates the burden on the skin of grooming salon staff, who are frequently exposed to cleansing agents.

However, the composition may contain a naturally derived amphoteric surfactant. Amphoteric surfactants cause less irritation and are safer than anionic and cationic surfactants. Although amphoteric surfactants alone do not provide sufficient cleansing efficacy, in the invention described in the present embodiment, the combination of amphoteric surfactants and calcium sodium phosphosilicate enables high cleansing power while maintaining safety.

Examples of naturally derived amphoteric surfactants include betaine-type amphoteric surfactants.

### {Other Components}

The composition may appropriately include, as needed, thickeners, wetting agents/moisturizers, skin conditioning agents, oils, pigments, dyes, salts, chelating agents, neutralizers, ultraviolet absorbers, preservatives, bactericides, purified water, natural water, deep-sea water, ethyl alcohol, and the like.

A thickener is an additive used to increase the viscosity of the composition, making it gel-like or cream-like. Examples of thickeners include “Aristoflex HMB” by Clariant Japan, synthetic polymers such as carboxyvinyl polymers, natural polymers such as pectin and xanthan gum, and inorganic materials such as fine-particle silica, bentonite, and hectorite.

A wetting agent/moisturizer is an additive used to provide moisturizing effects to the skin after using the composition. Examples of wetting agents/moisturizers include polyethylene glycol 600 (abbreviated as PEG 600), glycerin, amino acids, pyrrolidone carboxylic acid, lactic acid, mucopolysaccharides such as hyaluronic acid and chondroitin sulfate, and polyhydric alcohols such as glycerin, propylene glycol, butylene glycol, and sorbitol.

A skin conditioning agent is an additive used to improve the physical condition of the skin by providing emollient properties (reducing moisture evaporation from the skin to maintain hydration and soften the skin) and moisturizing effects to dry or damaged skin. Examples of skin conditioning agents include sugar derivatives such as hydroxyalkylated cyclodextrin, hydroxypropyl cellulose, acylated pullulan, stearoxy hydroxypropyl methylcellulose, and trehalose isostearate esters; hyaluronic acid, acetylated hyaluronic acid, collagen, carboxymethyl chitosan succinylamide, PPG-2 arginine, dilauroyl glutamate lysine sodium, amidinoproline, pyrrolidone carboxylic acid derivatives, glucosamine derivatives, alanyl glutamine, glyceryl-N-(2-hydroxyethyl) carbamate, glucamine derivatives, and water-soluble moisturizing oils such as PEG/PPG/polybutylene glycol-8/5/3 glycerin; lactones such as γ-docosalactone, gluconolactone, and erucalactone; ester oils such as cetyl 2-ethylhexanoate, di-2-heptylundecyl adipate, mixed fatty acid esters of diglycerin, isostearyl isostearate, octyldodecyl isostearate, phytosteryl isostearate, propylene glycol isostearate, isodecyl benzoate, isononyl polyglycerin isononanoate, esters of eicosanedioic acid/tetradecanedioic acid, cetyl octanoate, ethyl oleate, phytosteryl oleate, di-2-ethylhexyl succinate, diethylhexyl succinate, bis(diethylene glycol ethyl ether) succinate ester, diglycerin diisostearate, propylene glycol dicaprylate, neopentyl glycol dicaprate, tripropylene glycol dineopentanoate, dipentaerythritol and mixed fatty acid esters of hydroxystearic acid/stearic acid/rosin acid, dilinoleic acid di(phytosteryl/isostearyl/cetyl/steryl/behenyl), isocetyl stearate, stearyl stearate, hydrogenated castor oil stearate, diethyl sebacate, diethyl decanedioate, dextrin fatty acid ester, pentaerythrityl Tetraethylhexanoate, pentaerythritol tetra(2-ethylhexanoate), caprylic/capric triglyceride, tri-2-ethylhexanoyl glycerin, triethylhexanoin, isodecyl neopentanoate, isopropyl palmitate, ethylhexyl palmitate, octyl palmitate, hydrogenated castor oil hydroxystearate, phytosteryl isostearate, isopropyl myristate, octyldodecyl myristate, and myristyl myristate; ester oils such as diisostearyl malate, isopropyl lauroyl sarcosinate, (phytosteryl/behenyl/octyldodecyl/isostearyl) lauroyl glutamate, cholesterol, tocopheryl retinoate, allantoin, glycosaminoglycan, polyglutamic acid derivatives, nicotinic acid derivatives, phosphoserine, trehalose derivatives, tocopheryl phosphate, ascorbyl ethyl, ascorbyl-2-phosphate-6-fatty acid, whey derivatives, glycerophosphocholine, glucuronolactone, and glycerin-1-octadecyl urethane; and other components such as antioxidants, powders, urethane-based compounds, blood circulation promoters, local stimulants, antiandrogens, anti-seborrheic agents, anti-aging agents, emollients, keratolytic agents, and surfactant adjuvants.

### {Substantially not Containing Organic Solvents}

From the viewpoint of reducing skin damage, it is preferable that the composition substantially does not contain organic solvents.

From this viewpoint, it is preferable that the composition substantially does not contain organic solvents, such as hydrocarbon solvents including petroleum ether, n-hexane, cyclohexane, toluene, and benzene; halogenated hydrocarbons, including dichloromethane, chloroform, and carbon tetrachloride; ether solvents, including diethyl ether; ester solvents, including ethyl acetate; ketones including acetone; basic solvents, including pyridine; and monohydric or polyhydric alcohol solvents, including butanol, propanol, methanol, polyethylene glycol, propylene glycol, and butylene glycol. Furthermore, it is more preferable that the composition substantially does not contain isopropanol. Additionally, it is particularly preferable that the composition substantially does not contain ethanol.

### {Substantially not Containing Bactericidal Components or Antibiotics}

Up to now, methods such as bactericidal and cleansing treatments using shampoos containing bactericidal agents or antibiotics, as well as the application of topical medications (creams, gels, etc.) containing bactericidal agents or antibiotics, have been proposed.

In the case of shampoos containing bactericidal agents, since there is the possibility of the shampoo entering the eyes, nose, or mouth of the target animal, it is essential to reduce the burden on the animal. Additionally, when using shampoo to wash the target animal, there is a risk that the skin on the owner's hands and fingers may be significantly damaged. Similarly, in the case of topical medications, concerns arise regarding their effects on the animal and the increased burden on the owner.

Furthermore, in recent years, similar to humans, drug resistance to infectious diseases in dogs and cats has been increasing, leading to an increase in conditions for which antibiotics available at veterinary hospitals are ineffective; thus, from this perspective, it is essential that the composition substantially does not contain antibiotics.

According to the invention described in the present embodiment, since the composition substantially may not contain bactericidal components or antibiotics, the following effects are expected:
(1) A highly safe composition with low skin irritation and low oral toxicity can be provided.
(2) It poses no risks even if it enters the eyes, nose, or mouth of the animal and that does not damage human skin on the hands or fingers.
(3) It is effective against drug-resistant bacteria and that does not contribute to bacterial resistance.
(4) In addition to killing and eliminating bacteria that cause skin diseases, it is expected to improve the skin's moisturizing function.

### {pH of the Composition}

The pH of the composition is not particularly limited. From the viewpoint of promoting the softening of sebum (esters of glycerin and fatty acids) and skin keratin by sodium ions gradually released from calcium sodium phosphosilicate, the pH of the composition is preferably 8 or more, more preferably 8.5 or more, and even more preferably 9 or more.

Additionally, from the viewpoint of reducing skin damage caused by strong alkalinity, the pH of the composition is preferably less than 12, more preferably 11 or less, and even more preferably 10 or less.

### {Method for Cleansing Animals}

The method for cleansing animals according to the present embodiment includes a step of applying the aforementioned composition to the cleansing target site of an animal and a step of wiping off the composition applied to the cleansing target site. In this method, a water rinsing step is not required.

The type of animal is not particularly limited, but it is preferable that at least part of the surface of its body is covered with fur. Mammals generally have at least part of their body surface covered with fur. Among mammals, it is preferable that the animal is a pet (such as a dog or cat) that has a significant amount of fur on its body and is frequently handled in grooming salons, or a human with hair, with dogs or cats being particularly preferable.

In this method, since washing off the cleansing agent with running water is not required, the physical burden on the animal can be reduced, making it possible to use the composition even on elderly dogs or other animals with underlying conditions such as heart disease. Moreover, it reduces the labor burden on owners and grooming salons, allowing for daily use of the composition.

### EXAMPLES

The present invention is described in detail below with reference to examples; however, the invention is not limited thereto.

### <Experiment 1A> Verification of Emulsification Function (First Test)

### {Preparation of Animal Cleansing Agent Composition}

Animal cleansing agent compositions (sebum removers) for the examples and comparative examples were prepared according to the formulation shown in Table 1. The preparation was carried out by weighing each component under atmospheric pressure at room temperature, mixing by shaking, allowing the mixture to stand for 15 hours, and loading it into a cosmetic spray container. The pH of each composition was as indicated in Table 1.

TABLE 1

| | | | | | | | Comparative |
|---|---|---|---|---|---|---|---|
| Component | Example | | | | | | Example |
| Name | 1-1 | 1-2 | 1-3 | 1-4 | 1-5 | 1-6 | 1-1 |
| Calcium Sodium | 0.01 | 0.50 | 1.00 | 2.50 | 5.00 | 10.00 | — |
| Phosphosilicate | | | | | | | |
| Water | 99.99 | 99.50 | 99.00 | 97.50 | 95.00 | 90.00 | 100.00 |
| Total | 100.00 | 100.00 | 100.00 | 100.00 | 100.00 | 100.00 | 100.00 |
| pH | 10.80 | 11.38 | 11.57 | 11.54 | 12.01 | 12.53 | 7.24 |

Note:
The unit for each component in the formulation is mass parts.
Note:
Calcium sodium phosphosilicate is 45S5 bioactive glass (comprising 45% by weight silicon dioxide, 24.5% by weight calcium oxide, 24.5% by weight sodium oxide, and 6.0% by weight phosphorus pentoxide).

### {Evaluation}

To 100 g of the compositions of the examples and comparative examples, 5 g of olive oil was added, and the container was rotated slowly in a circular motion to mix the contents. After approximately 20 seconds of rotational mixing, the mixture was left to stand, and the state of the olive oil dispersion was observed. The results are shown in Table 2.

TABLE 2
Confirmation Results of Emulsification State

| | Emulsification State | Emulsification State After |
|---|---|---|
| | Immediately After Mixing | 2 Hours (Standing) |
| Comparative | The olive oil did not disperse | The water phase and oil phase |
| Example 1-1 | in the comparative example | (olive oil) were completely |
| | solution, and numerous oil | separated. |
| | droplets were observed in | |
| | the water. | |
| | When mixing was stopped, the | |
| | oil droplets quickly coalesced | |
| | and separated into a water phase | |
| | and an oil phase (olive oil). | |
| Examples | The olive oil dispersed in the | The milky water phase was |
| 1-1, 1-2, 1-3 | water phase immediately after | maintained (emulsified state). |
| | mixing, turning the water phase | Some of the olive oil separated |
| | milky. | as an oil phase. |
| | Some of the olive oil remained as | |
| | oil droplets, and when mixing | |
| | was stopped, they coalesced to | |
| | form an oil phase. | |
| Examples | The olive oil easily dispersed in | The olive oil was emulsified and |
| 1-4, 1-5, 1-6 | the water phase immediately after | dispersed in the water phase |
| | mixing, turning the water phase | (milky state). |
| | milky. | A small amount of olive oil |
| | A small amount of olive oil | separated as an oil phase on the |
| | remained as oil droplets, and | surface of the water phase. |
| | when mixing was stopped, they | |
| | coalesced to form a thin oil | |
| | phase on the surface. | |

As described in Comparative Example 1-1, oil-based components do not easily disperse in water; however, it was confirmed that the compositions of the examples, consisting of calcium sodium phosphosilicate and water, exhibited an emulsifying effect that allowed oil-based components, which do not inherently disperse in water, to be readily dispersed in water.

### <Experiment 1B> Verification of Emulsification Function (Second Test)

Following Table 1, calcium sodium phosphosilicate was removed from the compositions of the comparative example and the examples through suction filtration using a membrane filter with a pore size of 1 μm, thereby obtaining a calcium sodium phosphosilicate extract. A verification experiment was conducted to confirm the emulsification function of this calcium sodium phosphosilicate extract.

Specifically, 5 g of olive oil was added to 100 g of the composition from the examples, and the container was rotated slowly in a circular motion to mix the contents. After approximately 20 seconds of rotational mixing, the mixture was left to stand, and the state of the olive oil dispersion was observed. The results are shown in Table 3.

TABLE 3
Confirmation Results of Emulsification State

| Example and | | |
|---|---|---|
| Comparative | Emulsification State | Emulsification State After |
| Example | Immediately After Mixing | 2 Hours (Standing) |
| Extract from | The olive oil dispersed in the water | The milky water phase was |
| Example 1-1 | phase immediately after mixing, | maintained (emulsified state). |
| Extract from | turning the water phase milky. | Some of the olive oil separated |
| Example 1-2 | Some of the olive oil remained as | as an oil phase. |
| Extract from | oil droplets, and when mixing was | |
| Example 1-3 | stopped, they coalesced to form an | |
| | oil phase. | |
| Extract from | The olive oil easily dispersed in | The olive oil was emulsified and |
| Example 1-4 | the water phase immediately after | dispersed in the water phase |
| Extract from | mixing, turning the water phase | (milky state). |
| Example 1-5 | milky. | A small amount of olive oil |
| Extract from | A small amount of olive oil | separated as an oil phase on the |
| Example 1-6 | remained as oil droplets, and when | surface of the water phase. |
| | mixing was stopped, they | |
| | coalesced to form a thin oil phase | |
| | on the surface. | |

As shown in Table 3, it was confirmed that even the extract obtained by removing calcium sodium phosphosilicate from the compositions of the examples exhibited an emulsifying effect that allowed oil-based components, which do not inherently disperse in water, to be readily dispersed in water.

### <Experiment 1C> Test for Sebum Removal in Dogs

### {Cleansing Agents}

The compositions of Examples 1-1, 1-4, and 1-6, as well as the extract obtained by removing calcium sodium phosphosilicate from the composition of Example 1-4, and the composition of Comparative Example 1-1 were loaded into spray bottle containers to prepare sebum removal sprays.

### {Cleansing of Experimental Dogs}

The sebum removal spray was sprayed onto the neck area, between the toes, and the lower abdomen of experimental dogs that exhibited particularly high sebum levels, covering both the skin and fur. The sebum removal spray was applied until the fur was completely soaked. After application, the skin and fur of the experimental dogs were thoroughly massaged, and sebum removal was conducted by wiping with a dry wipe.

The treatment procedure was carried out by a professional groomer, and the effectiveness was evaluated as described in Table 4. The results are shown in Table 5.

TABLE 4

| Evaluation | | Evaluation Results | | |
|---|---|---|---|---|
| Criteria | | ⊚ | ◯ | X |
| 1 | Has stickiness been removed? | Significant | Moderate | None |
| 2 | Has odor been reduced? | Significant | Moderate | None |
| 3 | Has combing improved? | Significant | Moderate | None |
| 4 | Have dandruff and itching been reduced? | Significant | Moderate | None |
| 5 | Has the coat's shine improved? | Significant | Moderate | None |
| 6 | Was a moisturizing effect observed? | Significant | Moderate | None |
| 7 | Was the cleansing effect satisfactory? | Significant | Moderate | None |
| 8 | Were any side effects observed? | None | — | Present |

TABLE 5
Evaluation Results

| | | | Evaluation Results | | | | | | | |
|---|---|---|---|---|---|---|---|---|---|---|
| | Experimental Dog | | Criteria | | | | | | | |
| Sample | Type | Sex | 1 | 2 | 3 | 4 | 5 | 6 | 7 | 8 |
| Example 1-1 | Shih Tzu | F | ⊚ | ⊚ | ◯ | ⊚ | ⊚ | ◯ | ⊚ | N |
| | Pug | M | ⊚ | ◯ | ⊚ | ⊚ | ◯ | ⊚ | ⊚ | N |
| Example 1-4 | Shiba Inu | M | ⊚ | ⊚ | ◯ | ⊚ | ⊚ | ⊚ | ⊚ | N |
| | Shih Tzu | M | ⊚ | ◯ | ◯ | ⊚ | ⊚ | ◯ | ⊚ | N |
| Example 1-6 | Shepherd | M | ⊚ | ⊚ | ⊚ | ⊚ | ◯ | ⊚ | ⊚ | N |
| | Pug | F | ⊚ | ◯ | ⊚ | ⊚ | ⊚ | ◯ | ⊚ | N |
| Extract from | Mixed breed | F | ⊚ | ⊚ | ⊚ | ⊚ | ◯ | ⊚ | ⊚ | N |
| Example 1-4 | Shih Tzu | M | ⊚ | ⊚ | ◯ | ⊚ | ⊚ | ◯ | ◯ | N |
| Comparative | Shih Tzu | M | X | X | X | X | X | X | X | N |
| Example 1-1 | American Cocker Spaniel | F | X | X | X | X | X | X | X | N |

### <Experiment 2> Verification Test Using Shampoo Composition

### {Preparation of Shampoo Composition}

Following Tables 6 and 7, the components were measured under atmospheric pressure at room temperature, mixed by shaking, and then loaded into a cosmetic foam container to prepare the shampoo compositions for the examples and comparative examples.

TABLE 6

| | | | | | | | Comparative |
|---|---|---|---|---|---|---|---|
| | Example | | | | | | Example |
| Component Name | 2-1 | 2-2 | 2-3 | 2-4 | 2-5 | 2-6 | 2-1 |
| Calcium Sodium | 0.01 | 0.50 | 1.00 | 2.50 | 5.00 | 10.00 | |
| Phosphosilicate | | | | | | | |
| Water | 14.94 | 14.45 | 13.95 | 12.45 | 9.95 | 4.95 | 14.95 |
| Rebon HC-30W | 70.00 | 70.00 | 70.00 | 70.00 | 70.00 | 70.00 | 70.00 |
| Concentrated | 15.00 | 15.00 | 15.00 | 15.00 | 15.00 | 15.00 | 15.00 |
| Glycerin | | | | | | | |
| Tea Tree Oil | 0.05 | 0.05 | 0.05 | 0.05 | 0.05 | 0.05 | 0.05 |
| Total | 100.00 | 100.00 | 100.00 | 100.00 | 100.00 | 100.00 | 100.00 |

TABLE 7

| | Example | | |
|---|---|---|---|
| Component Name | 2-7 | 2-8 | 2-9 |
| Extract from | 44.95 | | |
| Example 1-2 | | | |
| Extract from | | 44.95 | |
| Example 1-4 | | | |
| Extract from | | | 44.95 |
| Example 1-6 | | | |
| Rebon HC-30W | 50.00 | 50.00 | 50.00 |
| Concentrated | 5.00 | 5.00 | 5.00 |
| Glycerin | | | |
| Tea Tree Oil | 0.05 | 0.05 | 0.05 |
| Total | 100.00 | 100.00 | 100.00 |

Note:
The unit for each component in the formulation is mass parts.
Note:
Calcium sodium phosphosilicate is 45S5 bioactive glass (comprising 45% by weight silicon dioxide, 24.5% by weight calcium oxide, 24.5% by weight sodium oxide, and 6.0% by weight phosphorus pentoxide).
Note:
Rebon HC-30W is an amphoteric surfactant (cocamidopropyl betaine aqueous solution; manufactured by Sanyo Chemical).
Note:
Concentrated glycerin is a moisturizing ingredient (manufactured by Sakamoto Yakuhin Kogyo Co., Ltd.).
Note:
Tea tree oil is a natural essential oil (fragrance; manufactured by Down Under Enterprises).

### {Verification of the Effectiveness of Shampoo Composition Using Experimental Dogs}

### [Washing of Experimental Dogs]

The neck area, the space between the toes, and the lower abdomen of experimental dogs with particularly high sebum levels were wetted with lukewarm water, and an appropriate amount of the shampoo composition was used to create foam and cleanse the dogs with thorough massaging. After leaving the foam on for five minutes, the shampoo composition was rinsed off thoroughly with lukewarm water and dried using a dryer.

The procedure was performed by a professional groomer, and the effectiveness was evaluated as shown in Table 8. The results are shown in Table 9.

TABLE 8

| Evaluation | | Evaluation Results | | |
|---|---|---|---|---|
| Criteria | | ⊚ | ◯ | X |
| 1 | Has stickiness been removed? | Significant | Moderate | None |
| 2 | Has odor been reduced? | Significant | Moderate | None |
| 3 | Has combing improved? | Significant | Moderate | None |
| 4 | Have dandruff and itching been reduced? | Significant | Moderate | None |
| 5 | Has the coat's shine improved? | Significant | Moderate | None |
| 6 | Was a moisturizing effect observed? | Significant | Moderate | None |
| 7 | Was the cleansing effect satisfactory? | Significant | Moderate | None |
| 8 | Were any side effects observed? | None | — | Present |

TABLE 9
Evaluation Results

| | | | Evaluation Results | | | | | | | |
|---|---|---|---|---|---|---|---|---|---|---|
| | Experimental Dog | | Criteria | | | | | | | |
| Sample | Type | Sex | 1 | 2 | 3 | 4 | 5 | 6 | 7 | 8 |
| Example 2-1 | Collie | F | ⊚ | ⊚ | ◯ | ◯ | ⊚ | ⊚ | ⊚ | N |
| | Mixed Breed | M | ⊚ | ⊚ | ⊚ | ⊚ | ◯ | ◯ | ⊚ | N |
| Example 2-2 | West Highland White | M | ⊚ | ⊚ | ⊚ | ◯ | ⊚ | ◯ | ⊚ | N |
| | Terrier | | | | | | | | | |
| | Shiba Inu | F | ⊚ | ⊚ | ◯ | ◯ | ⊚ | ⊚ | ⊚ | N |
| Example 2-3 | Toy Poodle | M | ⊚ | ⊚ | ⊚ | ⊚ | ⊚ | ◯ | ⊚ | N |
| | Shih Tzu | F | ⊚ | ⊚ | ◯ | ◯ | ◯ | ⊚ | ⊚ | N |
| Example 2-4 | Dachshund | F | ⊚ | ⊚ | ◯ | ◯ | ⊚ | ◯ | ⊚ | N |
| | Toy Poodle | F | ⊚ | ⊚ | ⊚ | ⊚ | ◯ | ⊚ | ⊚ | N |
| Example 2-5 | Golden Retriever | M | ⊚ | ⊚ | ⊚ | ◯ | ◯ | ⊚ | ⊚ | N |
| | Pug | M | ⊚ | ⊚ | ◯ | ◯ | ◯ | ◯ | ⊚ | N |
| Example 2-6 | Miniature Schnauzer | F | ⊚ | ⊚ | ◯ | ⊚ | ◯ | ◯ | ⊚ | N |
| | Dachshund | F | ⊚ | ⊚ | ◯ | ⊚ | ◯ | ◯ | ⊚ | N |
| Example 2-7 | Shiba Inu | M | ⊚ | ⊚ | ⊚ | ◯ | ⊚ | ◯ | ⊚ | N |
| | Toy Poodle | F | ⊚ | ⊚ | ◯ | ⊚ | ⊚ | ◯ | ⊚ | N |
| Example 2-8 | French Bulldog | M | ⊚ | ⊚ | ⊚ | ◯ | ⊚ | ⊚ | ⊚ | N |
| | Shih Tzu | F | ⊚ | ⊚ | ◯ | ⊚ | ◯ | ⊚ | ⊚ | N |
| Example 2-9 | Toy Poodle | F | ⊚ | ⊚ | ⊚ | ◯ | ◯ | ⊚ | ⊚ | N |
| | Pug | F | ⊚ | ⊚ | ◯ | ⊚ | ⊚ | ⊚ | ⊚ | N |
| Comparative | Shih Tzu | M | X | X | ◯ | X | ◯ | X | X | N |
| Example 2-1 | Golden Retriever | M | X | X | ◯ | ◯ | ◯ | X | X | N |

### <Experiment 3> Verification Test Using Mousse (Foam) Composition

### {Preparation of Mousse (Foam) Composition}

Following Tables 10 and 11, the components were measured under atmospheric pressure at room temperature, mixed by shaking, and then loaded into a cosmetic foam container to prepare the mousse (foam) compositions for the examples and comparative examples.

TABLE 10

| | | | | | | | Comparative |
|---|---|---|---|---|---|---|---|
| | Example | | | | | | Example |
| Component Name | 3-1 | 3-2 | 3-3 | 3-4 | 3-5 | 3-6 | 3-1 |
| Calcium Sodium | 0.01 | 0.50 | 1.00 | 2.50 | 5.00 | 10.00 | |
| Phosphosilicate | | | | | | | |
| Water | 97.94 | 97.45 | 96.95 | 95.45 | 92.95 | 87.95 | 97.95 |
| Soap Base | 2.00 | 2.00 | 2.00 | 2.00 | 2.00 | 2.00 | 2.00 |
| Tea Tree Oil | 0.05 | 0.05 | 0.05 | 0.05 | 0.05 | 0.05 | 0.05 |
| Total | 100.00 | 100.00 | 100.00 | 100.00 | 100.00 | 100.00 | 100.00 |

TABLE 11

| Component | Example | | |
|---|---|---|---|
| Name | 3-7 | 3-8 | 3-9 |
| Extract from | 97.95 | | |
| Example 1-2 | | | |
| Extract from | | 97.95 | |
| Example 1-4 | | | |
| Extract from | | | 97.95 |
| Example 1-6 | | | |
| Soap Base | 2.00 | 2.00 | 2.00 |
| Tea Tree Oil | 0.05 | 0.05 | 0.05 |
| Total | 100.00 | 100.00 | 100.00 |

Note: The unit for each component in the formulation is mass parts.
Note: Calcium sodium phosphosilicate is 45S5 bioactive glass (comprising 45% by weight silicon dioxide, 24.5% by weight calcium oxide, 24.5% by weight sodium oxide, and 6.0% by weight phosphorus pentoxide).
Note: Rebon HC-30W is an amphoteric surfactant (cocamidopropyl betaine aqueous solution; manufactured by Sanyo Chemical).
Note: Soap base is a composition containing glycerin, sodium stearate, sodium laurate, and sodium myristate (manufactured by Stephenson Personal Care, UK).
Note: Tea tree oil is a natural essential oil (fragrance; manufactured by Down Under Enterprises).

### {Verification of the Effectiveness of Mousse (Foam) Composition Using Experimental Dogs}

### [Washing of Experimental Dogs]

An appropriate amount of the mousse (foam) composition was massaged into the neck area, the space between the toes, and the lower abdomen of experimental dogs with particularly high sebum levels, ensuring thorough cleansing with massage. After leaving it on for five minutes, the composition was wiped off with a wipe and allowed to air dry.

The procedure was performed by a professional groomer, and the effectiveness was evaluated as shown in Table 12. The results are shown in Table 13.

TABLE 12

| Evaluation | | Evaluation Results | | |
|---|---|---|---|---|
| Criteria | | ⊚ | ◯ | X |
| 1 | Has stickiness been removed? | Significant | Moderate | None |
| 2 | Has odor been reduced? | Significant | Moderate | None |
| 3 | Has combing improved? | Significant | Moderate | None |
| 4 | Have dandruff and itching been reduced? | Significant | Moderate | None |
| 5 | Has the coat's shine improved? | Significant | Moderate | None |
| 6 | Was a moisturizing effect observed? | Significant | Moderate | None |
| 7 | Was the cleansing effect satisfactory? | Significant | Moderate | None |
| 8 | Were any side effects observed? | None | — | Present |

TABLE 13
Evaluation Results

| | | | Evaluation Results | | | | | | | |
|---|---|---|---|---|---|---|---|---|---|---|
| | Experimental Dog | | Criteria | | | | | | | |
| Composition | Type | Sex | 1 | 2 | 3 | 4 | 5 | 6 | 7 | 8 |
| Example 3-1 | Toy Poodle | M | ⊚ | ⊚ | ⊚ | ⊚ | ⊚ | ⊚ | ⊚ | N |
| | Mixed Breed | M | ⊚ | ◯ | ⊚ | ⊚ | ◯ | ◯ | ⊚ | N |
| Example 3-2 | Shih Tzu | F | ⊚ | ⊚ | ⊚ | ⊚ | ⊚ | ◯ | ⊚ | N |
| | Chihuahua | F | ⊚ | ⊚ | ◯ | ⊚ | ⊚ | ◯ | ⊚ | N |
| Example 3-3 | French Bulldog | M | ⊚ | ◯ | ⊚ | ⊚ | ⊚ | ◯ | ⊚ | N |
| | Pomeranian | M | ⊚ | ⊚ | ⊚ | ◯ | ⊚ | ◯ | ⊚ | N |
| Example 3-4 | Toy Poodle | M | ◯ | ◯ | ⊚ | ⊚ | ⊚ | ◯ | ⊚ | N |
| | French Bulldog | F | ⊚ | ⊚ | ⊚ | ◯ | ◯ | ◯ | ⊚ | N |
| Example 3-5 | Chihuahua | M | ⊚ | ◯ | ⊚ | ⊚ | ⊚ | ◯ | ⊚ | N |
| | Yorkshire Terrier | F | ◯ | ◯ | ◯ | ⊚ | ◯ | ◯ | ⊚ | N |
| Example 3-6 | Pomeranian | M | ⊚ | ⊚ | ◯ | ◯ | ◯ | ◯ | ⊚ | N |
| | Toy Poodle | F | ⊚ | ⊚ | ◯ | ◯ | ◯ | ◯ | ⊚ | N |
| Example 3-7 | Yorkshire Terrier | M | ⊚ | ⊚ | ◯ | ⊚ | ◯ | ◯ | ⊚ | N |
| | Toy Poodle | F | ⊚ | ◯ | ◯ | ⊚ | ◯ | ◯ | ⊚ | N |
| Example 3-8 | Toy Poodle | F | ⊚ | ⊚ | ⊚ | ◯ | ⊚ | ◯ | ⊚ | N |
| | Chihuahua | F | ⊚ | ◯ | ⊚ | ⊚ | ⊚ | ⊚ | ⊚ | N |
| Example 3-9 | Toy Poodle | F | ⊚ | ⊚ | ⊚ | ⊚ | ◯ | ⊚ | ⊚ | N |
| | Chihuahua | M | ⊚ | ⊚ | ⊚ | ⊚ | ⊚ | ⊚ | ⊚ | N |
| Comparative | Pug | M | X | X | ◯ | X | ◯ | X | X | N |
| Example 3-1 | Toy Poodle | F | X | ◯ | X | X | X | X | X | N |

1. A cleansing agent composition for dogs and cats comprising calcium sodium phosphosilicate in an amount of 0.01 mass parts or more and 10.0 mass parts or less, and water, having a pH of 9 or more and less than 13.

2. The cleansing agent composition according to claim 1, wherein the calcium sodium phosphosilicate comprises a bioactive glass containing silicon dioxide, calcium oxide, sodium oxide, and phosphorus pentoxide, and a bioactive glass extract obtained by removing the bioactive glass from a bioactive glass-containing liquid comprising the bioactive glass and an aqueous solvent.

3. (canceled)

4. The cleansing agent composition according to claim 1, wherein the composition substantially does not contain synthetic surfactants.

5. The cleansing agent composition according to claim 1, further comprising a naturally derived amphoteric surfactant.

6. A method for cleansing a dog or a cat, comprising a step of applying the composition according to claim 1 to a cleansing target site of the dog or the cat, and a step of wiping off the composition applied to the cleansing target site, wherein the method does not require a water rinsing step.

7. A method for cleansing a dog or a cat, comprising a step of applying the composition according to claim 2 to a cleansing target site of the dog or the cat, and a step of wiping off the composition applied to the cleansing target site, wherein the method does not require a water rinsing step.

8. A method for cleansing a dog or a cat, comprising a step of applying the composition according to claim 4 to a cleansing target site of the dog or the cat, and a step of wiping off the composition applied to the cleansing target site, wherein the method does not require a water rinsing step.

9. A method for cleansing a dog or a cat, comprising a step of applying the composition according to claim 5 to a cleansing target site of the dog or the cat, and a step of wiping off the composition applied to the cleansing target site, wherein the method does not require a water rinsing step.
